SEQUENCE LISTING (28 sequences)

SEQ ID NO: 1 - DNA, 58 bp, Bacillus subtilis
  source 1..58: mol_type = other DNA
acagaatagtcttttaagtaagtctactctgaatttttttaaaaggagagggtaaaga

SEQ ID NO: 2 - DNA, 87 bp, Bacillus subtilis
  source 1..87: mol_type = other DNA
gtgagaagcaaaaaattgtggatcagcttgttgtttgcgttaacgttaatctttacgatggcgttcagcaacatgtctgcgcaggct

SEQ ID NO: 3 - protein, 29 aa, Bacillus subtilis
VRSKKLWISLLFALTLIFTMAFSNMSAQA

SEQ ID NO: 4 - DNA, 252 bp, Bacillus clausii
  source 1..252: mol_type = other DNA
gcggaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatg

SEQ ID NO: 5 - protein, 84 aa, Bacillus clausii
AEEAKEKYLIGFNEQEAVSEFVEQVEANDEVAILSEEEEVEIELLHEFETIPVLSVELSPEDVDALELDPAISYIEEDAEVTTM

SEQ ID NO: 6 - DNA, 807 bp, Bacillus clausii
  source 1..807: mol_type = other DNA
gcgcaatcggtaccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggctgggacgattgctgctttaaacaattcgattggcgttcttggcgtagcaccgaacgcggaactatacgctgttaaagtattaggggcgagcggtatgggttcggtcagctcgattgcccaaggattggaatgggcagggaacaatgttatgcacgttgctaatttgagtttaggactgcaggcaccaagtgccacacttgagcaagctgttaatagcgcgacttctagaggcgttcttgttgtagcggcatctggcaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtccaaatccgcaatcatctaaagaatacggcaacgagcttaggaagcacgaacttgtatggaagcggacttgtcaatgcagaagcggcaacacgt

SEQ ID NO: 7 - protein, 269 aa, Bacillus clausii
AQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGMGSVSSIAQGLEWAGNNVMHVANLSLGLQAPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 8 - DNA, 247 bp, Bacillus amyloliquefaciens
  source 1..247: mol_type = other DNA
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 9 - DNA, 1008 bp, Bacillus subtilis
  source 1..1008: mol_type = other DNA
tgttggtgattctcatattatggtcgtttgccgcatggtccaccggtccgacacagcaatttcacctggctacaatagaaccggaaatgtcaggtgttttgcttagcatgaatcagtcaatgatgcaattcgccatggcggtcggcgcaggtataggaggggtttttgtggaaaacgtatcattggcctcgattacctgggttggtgcgttaggggttatgattgcaattattgcatcattgctgattttcaattcgcaaccgaaacaagcgctaaaagatatcaatcaataatttcgaaagttctaacatccgctcgttatacaagcgggtgttttttttagcgtagtcagtccatgcatgagtcttccaagggccgacatcgttctgtatgtgtcgaaggtgcactgaagctgaatgagatttcttaaggctttgccgcggcgagctgaagtacagtacgattgccttgatcgaacactggaaacacaagagaacgtaaacctgaacatccgcgtaaacgtcaaggaagttgccacttggggagtaaacacttgcatcatctcgctgaaaggcctagacaatgcggatgacagattcgtattgccggaagtaaacacagcgcttgctctgtttccgttgtcgattgcagctgattgcttactaatgctgccctgcatctcggttgtgatgtcgataagcagcgtaatgaagagtgttactgtggagtaagatgtttaacccctctggatattttagccgaaggggtttttagtatagatatttgatttaaattacaggagacttcattcatttaaccaaagtcattgctttcttaatcactaaagtttatttgaaaataatctcttgatttaatttcctcgaagagattttttgtcaatctattaggcatcagaatttttataacataatggaccgtctttttgacgttttgtttatagaacaagaaaatattcaaaacataagtggaaaattaggggtgagctcc

SEQ ID NO: 10 - DNA, 765 bp, Bacillus subtilis
  source 1..765: mol_type = other DNA
ggtccttcctataagcaggaaggttttttaatgaaatatttttttattagtaggtaaattcatcaaatgttctgctatgctccaaatgtacacctttccgtaagggcaaagtcaaatgtgaaaaaaaactgttccaaagttaatcagaaatttattttcatattgtatccttcggtatcaagtgaaaggagcatcatatgaaaacattatggaaagtcctcaaaattgtttttgtcagcttggctgctttggttttgcttgtatccgtctcggtatttatttatcaccatttccagctaaataaggaggcggcactgctgaaaggtaaaggcacagtagtcgatgttgacggtaaaaagatgaatgtgtatcaagagggaagcgggaaggatacgtttgtgtttatgtccggttcgggtattgctgcgcctgcttatgaaatgaaggggctgtacagcaagttttcaaaagaaaataagattgctgttgtagatcgggcaggttatggatacagtgaagtgtctcacgatgacagagatattgatacggtattggaacagacgaggaaagcgcttatgaaaagcggaaataagcctccttatattttaatgcctcattcgatatccgggattgaagcgatgtattgggcacagaaatatcctaaggaaatcaaggccattattgcgatggatattggattgccccagcagtacgtcacgtataaattgagcggagttgaccgattgaaagtgagagggttccacc

SEQ ID NO: 11 - DNA, 984 bp, Bacillus subtilis
  source 1..984: mol_type = other DNA
accaggcaccgaagcgtattcacttaatccgccatcacgtgagacaccgagctcatagcttgtcgcgatcacctcatccccctccgcaaaacgcggatcattggaagagacgaccgtacccgcagcatcaatgcctaaaataagcggatactctctgacgatattgcctcctgcttttccggccagaccatctttgtaattaatgccggaataagcaactttaatcaggacaccatccttcggcaaatcctctgttgatatggttttcacatggactgaaacatcatcggcatttttttctgcctgcaaggcttgaaataacgttgacattcggcacactccttttcatttatatcgtaaccgaagaacgttcaaaaaaccaaatcatcaagccgccattttcacttcgccggcacattgagacaataatggacaaatccggtatcctcttcatagccgttttgctcatacaagcttcttgccttccggttgtggtgctcagtctgaagtgttaaacattttgccccgttttgccctgcataatcctttgcggcagaaagcagccggccgccggctccctttgtacgcgcatgaggaacgacaaataagtcatttaatatgtatatccttttcattgacacagaagaaaacgttggatagagctgggtaaagcctatgaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacg

SEQ ID NO: 12 - DNA, 1170 bp, Bacillus subtilis
  source 1..1170: mol_type = other DNA
atgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaa

SEQ ID NO: 13 - DNA, 1001 bp, Bacillus subtilis
  source 1..1001: mol_type = other DNA
tttatttgtcagcatcctgatgttccggcgcattctcttctttctccgcatgttgaatccgttccatgatcgacggatggctgcctctgaaaatcttcacaagcaccggaggatcaacctggctcagccccgtcacggccaaatcctgaaacgttttaacagcggcttctctgttctctgtcaactcgatcccatactggtcagccttattctcctgataacgcgagacagcattagaaaaaggcgtaaccgcaaagctcaaaacagaaaacaaaagcaataacagcggaagtgccgcaagatcatgccgcccttctaaatgaaacatgctgcgggttaggcgaaccgtccgcttgtaaagcttatcaatgacataaaatccggcgagcgacacgagcaaatagccagccagaccgatgtaaacgtgcttcatgacataatggcccatttcgtggcccataataaacagaatttctgaatcgtcaagtttgttcagcgtcgtatcccacaatacaatccgtttattggccccaattcctgtaacataggcattcagcgcatttgttttttctgacatgttcacttcatatacatggtcagccggaatattggcttcatctgccagctctaaaattttgctttcaagctctttgtttttcagcggataaaaatcattgtataaaggatcgataatgaccggctgaataaaaaacagaaacagcgaaaacggcactgttaacagccaggcgtataaccaccattttttttcatgccttttgatcagccaataaaaaacgagaacgcaaagcgtaaagattggaaagctgatccaaaagctgataacctgatccttagcccagctggccgttgtctgtgtggaaatgttatagtcaagcgatacttgatagcctatccaatctaaaggcagcgtcaccaatgttgtaatcagcgaaagcacaaacacaaaaccaacggtctgcaaaaaccgaaaaggcacggcc

SEQ ID NO: 14 - DNA, 1071 bp, Bacillus subtilis
  source 1..1071: mol_type = other DNA
ttgaataagcttattgaacgagaaaaaactgtatattataaggaaaagcccgacccgtcttccttggggtttggacaatattttacagattatatgtttgtgatggactacgaagaggggattggatggcatcatccgagaattgcgccgtacgcaccgcttacgcttgatccgtcttcatctgtttttcattacggccaggctgtttttgaaggattaaaagcatacagaacagacgacggcagggtgctgctgttccgtccggatcaaaatatcaaacggctgaacagatcgtgtgagcgcatgagcatgccccctttagacgaagagctggtgcttgaggcattgacgcaattagttgagctggagaaagattgggttccaaaggaaaaaggaacgtcactgtatattcgtccttttgtcattgccacagaaccgagtctcggtgtgaaggcatccaggagctatacatttatgatcgtgctttcgcctgtcggctcctattatggcgacgatcagctgaagccggttagaatctatgtcgaagatgagtatgtgagggcggtcaacggaggagtcgggtttgcaaaaacggctggaaactatgccgccagtcttcaggcacagcggaaagcgaatgaactgggctatgaccaggtactgtggctggacgccatcgaaaagaaatatgtggaagaagtagggagcatgaacatctttttcgtcataaacggggaagctgtcacacctgctttaagcggaagcattttaagcggggttacacgtgcgtctgcgattgaattgattcgaagctggggcattccggttcgtgaagagagaatatcgattgatgaggtgtatgcggcctctgcacgcggagaattgacagaggtctttggcacaggcacggcagcagtcgttacgcctgtcggtgaactcaacatccatggaaaaacggtgattgtaggcgacgggcaaatcggggacctctcgaaaaagctgtatgaaacgataacagatattcagcttggcaaggtaaaaggcccgtttaactggacagtggaagtgtga

SEQ ID NO: 15 - protein, 356 aa, Bacillus subtilis
MNKLIEREKTVYYKEKPDPSSLGFGQYFTDYMFVMDYEEGIGWHHPRIAPYAPLTLDPSSSVFHYGQAVFEGLKAYRTDDGRVLLFRPDQNIKRLNRSCERMSMPPLDEELVLEALTQLVELEKDWVPKEKGTSLYIRPFVIATEPSLGVKASRSYTFMIVLSPVGSYYGDDQLKPVRIYVEDEYVRAVNGGVGFAKTAGNYAASLQAQRKANELGYDQVLWLDAIEKKYVEEVGSMNIFFVINGEAVTPALSGSILSGVTRASAIELIRSWGIPVREERISIDEVYAASARGELTEVFGTGTAAVVTPVGELNIHGKTVIVGDGQIGDLSKKLYETITDIQLGKVKGPFNWTVEV

SEQ ID NO: 16 - DNA, 81 bp, Bacillus subtilis
  source 1..81: mol_type = other DNA
atcaaacagccagaataaaactggctgttttctttaatttcagcttcatctgacaatgaatgtggtgtataatagctgatg

SEQ ID NO: 17 - DNA, 127 bp, Bacillus subtilis
  source 1..127: mol_type = other DNA
atcctaaagaatattctgaaatttataggtgtgaagcggcgtattattaaaggaaaggctgtatactttttgcagctttaaaaagactgaatatttaaacaattatgtgaaaggtgtgctgattaga

SEQ ID NO: 18 - DNA, 127 bp, Bacillus subtilis
  source 1..127: mol_type = other DNA
atcctaaagaatattctgaaatttataggtgtgaagcggcgtattattaaaggaaaggctgtatactttttgtagctttaaaaagactgaatatttaaacaattatgtgaaaggtgtgctgattaga

SEQ ID NO: 19 - DNA, 23 bp, synthetic construct
  source 1..23: mol_type = other DNA
tgaagccggttagaatctatgtc

SEQ ID NO: 20 - DNA, 21 bp, synthetic construct
  source 1..21: mol_type = other DNA
ctggtcatagcccagttcatt

SEQ ID NO: 21 - DNA, 21 bp, synthetic construct
  source 1..21: mol_type = other DNA
tggcggcatagtttccagccg

SEQ ID NO: 22 - DNA, 19 bp, synthetic construct
  source 1..19: mol_type = other DNA
cgatcggaaagcttgctca

SEQ ID NO: 23 - DNA, 20 bp, synthetic construct
  source 1..20: mol_type = other DNA
ccgtctgcttaatgacaggc

SEQ ID NO: 24 - DNA, 22 bp, synthetic construct
  source 1..22: mol_type = other DNA
tccagctgttcaatggctcccg

SEQ ID NO: 25 - DNA, 1279 bp, Bacillus subtilis
  source 1..1279: mol_type = other DNA
atcaaacagccagaataaaactggctgttttctttaatttcagcttcatctgacaatgaatgtggtgtataatagctgatgatcctaaagaatattctgaaatttataggtgtgaagcggcgtattattaaaggaaaggctgtatactttttgcagctttaaaaagactgaatatttaaacaattatgtgaaaggtgtgctgattagattgaataagcttattgaacgagaaaaaactgtatattataaggaaaagcccgacccgtcttccttggggtttggacaatattttacagattatatgtttgtgatggactacgaagaggggattggatggcatcatccgagaattgcgccgtacgcaccgcttacgcttgatccgtcttcatctgtttttcattacggccaggctgtttttgaaggattaaaagcatacagaacagacgacggcagggtgctgctgttccgtccggatcaaaatatcaaacggctgaacagatcgtgtgagcgcatgagcatgccccctttagacgaagagctggtgcttgaggcattgacgcaattagttgagctggagaaagattgggttccaaaggaaaaaggaacgtcactgtatattcgtccttttgtcattgccacagaaccgagtctcggtgtgaaggcatccaggagctatacatttatgatcgtgctttcgcctgtcggctcctattatggcgacgatcagctgaagccggttagaatctatgtcgaagatgagtatgtgagggcggtcaacggaggagtcgggtttgcaaaaacggctggaaactatgccgccagtcttcaggcacagcggaaagcgaatgaactgggctatgaccaggtactgtggctggacgccatcgaaaagaaatatgtggaagaagtagggagcatgaacatctttttcgtcataaacggggaagctgtcacacctgctttaagcggaagcattttaagcggggttacacgtgcgtctgcgattgaattgattcgaagctggggcattccggttcgtgaagagagaatatcgattgatgaggtgtatgcggcctctgcacgcggagaattgacagaggtctttggcacaggcacggcagcagtcgttacgcctgtcggtgaactcaacatccatggaaaaacggtgattgtaggcgacgggcaaatcggggacctctcgaaaaagctgtatgaaacgataacagatattcagcttggcaaggtaaaaggcccgtttaactggacagtggaagtgtga

SEQ ID NO: 26 - DNA, 74 bp, Bacillus subtilis
  source 1..74: mol_type = other DNA
taaatccttgacgagcaagggattgacgctttaaaatgcttgatatggctttttatatgtgttactctacatac

SEQ ID NO: 27 - DNA, 1308 bp, synthetic construct
  source 1..1308: mol_type = other DNA
taaatccttgacgagcaagggattgacgctttaaaatgcttgatatggctttttatatgtgttactctacatacatcctaaagaatattctgaaatttataggtgtgaagcggcgtattattaaaggaaaggctgtatactttttgcagctttaaaaagactgaatatttaaacaattatgtgaaaggtgtgctgattagattgaataagcttattgaacgagaaaaaactgtatattataaggaaaagcccgacccgtcttccttggggtttggacaatattttacagattatatgtttgtgatggactacgaagaggggattggatggcatcatccgagaattgcgccgtacgcaccgcttacgcttgatccgtcttcatctgtttttcattacggccaggctgtttttgaaggattaaaagcatacagaacagacgacggcagggtgctgctgttccgtccggatcaaaatatcaaacggctgaacagatcgtgtgagcgcatgagcatgccccctttagacgaagagctggtgcttgaggcattgacgcaattagttgagctggagaaagattgggttccaaaggaaaaaggaacgtcactgtatattcgtccttttgtcattgccacagaaccgagtctcggtgtgaaggcatccaggagctatacatttatgatcgtgctttcgcctgtcggctcctattatggcgacgatcagctgaagccggttagaatctatgtcgaagatgagtatgtgagggcggtcaacggaggagtcgggtttgcaaaaacggctggaaactatgccgccagtcttcaggcacagcggaaagcgaatgaactgggctatgaccaggtactgtggctggacgccatcgaaaagaaatatgtggaagaagtagggagcatgaacatctttttcgtcataaacggggaagctgtcacacctgctttaagcggaagcattttaagcggggttacacgtgcgtctgcgattgaattgattcgaagctggggcattccggttcgtgaagagagaatatcgattgatgaggtgtatgcggcctctgcacgcggagaattgacagaggtctttggcacaggcacggcagcagtcgttacgcctgtcggtgaactcaacatccatggaaaaacggtgattgtaggcgacgggcaaatcggggacctctcgaaaaagctgtatgaaacgataacagatattcagcttggcaaggtaaaaggcccgtttaactggacagtggaagtgtgaaaatcgaaaaagaacctgcccggaggcaggttcttt

SEQ ID NO: 28 - DNA, 1308 bp, synthetic construct
  source 1..1308: mol_type = other DNA
taaatccttgacgagcaagggattgacgctttaaaatgcttgatatggctttttatatgtgttactctacatacatcctaaagaatattctgaaatttataggtgtgaagcggcgtattattaaaggaaaggctgtatactttttgtagctttaaaaagactgaatatttaaacaattatgtgaaaggtgtgctgattagattgaataagcttattgaacgagaaaaaactgtatattataaggaaaagcccgacccgtcttccttggggtttggacaatattttacagattatatgtttgtgatggactacgaagaggggattggatggcatcatccgagaattgcgccgtacgcaccgcttacgcttgatccgtcttcatctgtttttcattacggccaggctgtttttgaaggattaaaagcatacagaacagacgacggcagggtgctgctgttccgtccggatcaaaatatcaaacggctgaacagatcgtgtgagcgcatgagcatgccccctttagacgaagagctggtgcttgaggcattgacgcaattagttgagctggagaaagattgggttccaaaggaaaaaggaacgtcactgtatattcgtccttttgtcattgccacagaaccgagtctcggtgtgaaggcatccaggagctatacatttatgatcgtgctttcgcctgtcggctcctattatggcgacgatcagctgaagccggttagaatctatgtcgaagatgagtatgtgagggcggtcaacggaggagtcgggtttgcaaaaacggctggaaactatgccgccagtcttcaggcacagcggaaagcgaatgaactgggctatgaccaggtactgtggctggacgccatcgaaaagaaatatgtggaagaagtagggagcatgaacatctttttcgtcataaacggggaagctgtcacacctgctttaagcggaagcattttaagcggggttacacgtgcgtctgcgattgaattgattcgaagctggggcattccggttcgtgaagagagaatatcgattgatgaggtgtatgcggcctctgcacgcggagaattgacagaggtctttggcacaggcacggcagcagtcgttacgcctgtcggtgaactcaacatccatggaaaaacggtgattgtaggcgacgggcaaatcggggacctctcgaaaaagctgtatgaaacgataacagatattcagcttggcaaggtaaaaggcccgtttaactggacagtggaagtgtgaaaatcgaaaaagaacctgcccggaggcaggttcttt

(19) United States
(12) Patent Application Publication
(10) Pub. No.: US 2026/0132387 A1
(43) Pub. Date: May 14, 2026

(54) COMPOSITIONS AND METHODS FOR ENHANCED PROTEIN PRODUCTION IN BACILLUS CELLS

(71) Applicant: DANISCO US INC., Palo Alto, CA (US)

(72) Inventors: Cristina Bongiorni, Fremont, CA (US); Gopal K. Chotani, Cupertino, CA (US); Shannon Del Chase, Mountain View, CA (US); Chao Zhu, Mountain View, CA (US)

(21) Appl. No.: 19/121,647

(22) PCT Filed: Oct. 13, 2023
(86) PCT No.: PCT/US2023/076839

§ 371 Date: Apr. 16, 2025

Related U.S. Application Data
(60) Provisional application No. 63/380,706, filed on Oct. 24, 2022.

(51) Int. Cl. C12N 9/10; C12P 21/00

(52) U.S. Cl. CPC C12N 9/1096; C12P 21/00; C12Y 206/01042

(57) ABSTRACT

Certain embodiments of the disclosure are related to, inter alia, mutant and/or modified Bacillus cells (strains) comprising enhanced protein productivity phenotypes, mutant and/or modified cells comprising enhanced/increased ilvE messenger RNA (mRNA) levels, mutant and/or modified cells comprising enhanced/increased carbon yields (carbon yield efficiencies) of heterologous proteins produced and the like. As generally described herein, certain embodiments of the disclosure are related to mutant and/or modified Bacillus strains comprising variant ilvE genes, which mutant and/or modified Bacillus strains are particularly useful for the enhanced production of proteins of interests.

### FIELD

The present disclosure is generally related to the fields of bacteriology, microbiology, genetics, molecular biology, enzymology, industrial protein production the like. Certain embodiments of the disclosure are related to Bacillus sp. strains comprising enhanced protein productivity phenotypes, compositions and methods for constructing recombinant Bacillus sp. strains, and the like.

### CROSS REFERENCE TO RELATED APPLICATIONS

This application claims benefit to U.S. Provisional Patent Application No. 63/380,706, filed Oct. 24, 2022, which is incorporated herein by referenced in its entirety.

### REFERENCE TO A SEQUENCE LISTING

The contents of the electronic submission of the text file Sequence Listing, named “NB41976-WO-PCT_SequenceListing.xml” was created on Oct. 2, 2023 and is 37 KB in size, which is hereby incorporated by reference in its entirety.

### BACKGROUND

Gram-positive bacteria such as Bacillus subtilis, Bacillus lichemformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, Bacillus sp. host cells are well known for their production of enzymes (e.g., amylases, cellulases, mannanases, pectate lysases, proteases, pullulanases, etc.) necessary for food, textile, laundry, medical instrument cleaning, pharmaceutical industries and the like. Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority (EFSA), and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) via microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield and the like are quite significant when the protein is produced in large industrial quantities. For example, the expression of many heterologous proteins can still be challenging and unpredictable with respect to yield and the like. As described hereinafter, the present disclosure is related to the highly desirable and unmet needs for obtaining and constructing Bacillus sp. cells (e.g., protein production hosts) having enhanced protein production capabilities.

### SUMMARY

As generally described herein, certain embodiments of the disclosure are related to, inter alia, variant ilvE genes, variant ilvE gene 5′-untranslated region (5′-UTR) sequences, mutant Bacillus strains comprising variant ilvE gene sequences, recombinant (genetically modified) Bacillus strains comprising variant ilvE gene sequences, mutant and/or recombinant Bacillus strains comprising variant ilvE gene sequences and expressing/producing one or more proteins of interest, methods and compositions for constructing recombinant Bacillus strains comprising variant ilvE gene sequences, expression cassettes encoding proteins of interest, methods and compositions for cultivating recombinant Bacillus strains comprising variant ilvE gene sequences for the enhanced production of proteins of interest and the like. More particularly, as described hereinafter, the novel mutant and/or recombinant Bacillus cells of disclosure are particularly useful for the production of proteins of interests when cultivated under suitable conditions.

Certain embodiments of the disclosure are therefore related to variant ilvE genes comprising a single nucleotide polymorphism (SNP) mutation in the 5-untranslated region (5′-UTR) of the ilvE gene. In one or more embodiments, variant ilvE genes of the disclosure encode functional IlvE proteins. In certain other embodiments, the disclosure provides synthetic ilvE gene constructs comprising in the 5′ to 3′ direction, a heterologous promoter sequence operably linked to a mutant ilvE 5′-UTR sequence operably linked to an ilvE gene coding sequence (CDS) encoding a functional IlvE protein. In other embodiments, the disclosure is related to mutant Bacillus subtilis strains comprising a variant ilvE gene having a SNP in the 5′-untranslated region (5′-UTR) of the ilvE gene. In other one or more embodiments, mutant and/or recombinant B. subtilis cells of the disclosure produce one or more proteins of interest. In other related embodiments, mutant and/or recombinant B. subtilis cells producing one or more proteins of interest comprise enhanced carbon yield phenotypes relative to control B. subtilis cells producing the same one or more proteins of interest and comprising a wild-type ilvE gene, when the mutant and control cells are fermented under suitable conditions. In certain related one or more embodiments, the disclosure provides mutant and/or modified Bacillus cells (strains) comprising enhanced protein productivity phenotypes, mutant and/or modified cells comprising enhanced/increased ilvE messenger RNA (mRNA) levels, mutant and/or modified cells comprising enhanced/increased carbon yields (carbon yield efficiencies) of heterologous proteins produced and the like.

In other embodiments, the disclosure is related to methods for increasing ilvE messenger RNA (mRNA) levels in recombinant B. subtilis cells, the methods generally comprising obtaining a parental B. subtilis cell having a wild-type (WT) ilvE gene and replacing the WT ilvE gene with a variant ilvE gene, wherein the variant ilvE gene comprises a SNP mutation in the 5′-untranslated region (5′-UTR) of the ilvE gene, and fermenting the parental and recombinant cells for at least about sixteen hours under suitable conditions, wherein the recombinant cell comprises increased levels of ilvE mRNA as compared to the parental cell. In other embodiments, the disclosure is related to methods for increasing carbon yields of heterologous proteins produced in recombinant B. subtilis cells, the methods comprising obtaining or constructing a parental B. subtilis cell producing a heterologous protein of interest (POI) and comprising a WT ilvE gene, and replacing the WT ilvE gene with a variant ilvE gene comprising a SNP mutation in the 5′-untranslated region (5′-UTR) of the ilvE gene, and fermenting the parental and recombinant cells for at least about sixteen hours under suitable conditions for the production of the POI, wherein the recombinant cells comprise an increased carbon yield efficiency of the POI produced as compared to the parental cell.

### BRIEF DESCRIPTION OF DRAWINGS

FIG. 1 shows the DNA sequence of the wild-type (WT) B. subtilis ilvE 5′-UTR (FIG. JA) and the mutant B. subtilis ilvE 5′-UTR (FIG. 1B). For example, as shown in FIG. 1A, the WT ilvE 5′-UTR sequence comprises a cytosine (C) at nucleotide position 73 and the mutant ilvE 5′-UTR, as shown in FIG. 1B, comprises a thymine (T) at nucleotide position 73, wherein the C and T nucleotides at position are presented in bold, double underlined in FIG. 1. Thus, as presented in FIG. 1, the WT B. subtilis ilvE 5′-UTR sequence comprises SEQ ID NO: 17 (FIG. JA) and the mutant ilvE 5′-UTR sequence comprises SEQ ID NO: 18 (FIG. 1B).

FIG. 2 presents a schematic map showing nucleotide position 73 (+73) of the mutant ilvE 5′-UTR sequence (SEQ ID NO: 18). In particular, the WT (SEQ ID NO: 17) and mutant (SEQ ID NO: 18) ilvE 5′-UTR sequences presented in FIG. 1 are numbered in the 5′ to 3′ direction, wherein nucleotide position 1 (+1) is the first nucleotide position of the 5′ untranslated region identified as transcription start site. In particular, as shown in FIG. 2, the C>T mutation at position 73 in mutant ilvE 5′-UTR is located near the CodY binding motif 2, and a putative binding motif 5.

FIG. 3 shows the nucleic acid sequences of the wild-type (WT) ilvE promoter (FIG. 3A; SEQ ID NO: 16), the WT ilvE 5′-UTR (FIG. 3B; SEQ ID NO: 17), the WT ilvE gene coding sequence (FIG. 3C; SEQ ID NO: 14) and the WT ilvE gene (FIG. 3D; SEQ ID NO: 25). As presented in FIG. 3D in the 5′ to 3′ direction, the WT ilvE gene (SEQ ID NO: 25) comprises the WT ilvE promoter (italicized nucleotides; SEQ ID NO: 16), the WT ilvE 5′-UTR (bold nucleotides; SEQ ID NO: 17) and the WT ilvE gene CDS (underlined nucleotides; SEQ ID NO: 14). Likewise, FIG. 3 shows the amino acid sequence of the native (mature) IlvE protein (FIG. 3E; SEQ ID NO: 15) encoded by the WT ilvE gene CDS (SEQ ID NO: 14).

FIG. 4 presents data from real-time qPCR (RT qPCR) analysis of Bacillus Strain A (comprising mutated ilvE 5′-UTR) relative to Bacillus Strain B (comprising WT ilvE 5′-UTR), as described below in Example 2. In particular, the data presented in FIG. 4 show the results of the RT qPCR in a time course experiment, wherein the 2-1-AC_{T} method (Livak and Schrmittgen, 2001) was used to calculate the log-fold changes between the housekeeping ftsY gene and the ilvE gene. As shown in FIG. 4, the bars and values represent the fold changes in ilvE mRNA of B. subtilis strain A (mutant ilvE 5′-UTR) versus isogenic B. subtilis strain B (WT ilvE 5′-UTR) at 16, 24 and 32 hour fermentation time points.

### BRIEF DESCRIPTION OF THE BIOLOGICAL SEQUENCES

SEQ ID NO: 1 is a nucleotide (DNA) sequence comprising a wild-type B. subtilis aprE 5′-UTR sequence.

SEQ ID NO: 2 is a wild-type DNA sequence encoding a native B. subtilis aprE signal sequence.

SEQ ID NO: 3 is the amino acid sequence of the native B. subtilis aprE signal sequence encoded by SEQ ID NO: 2.

SEQ ID NO: 4 is a DNA sequence encoding a native B. clausii GG36 Pro region sequence.

SEQ ID NO: 5 is the amino acid sequence of the native B. clausii GG36 Pro region sequence encoded by SEQ ID NO: 4.

SEQ ID NO: 6 is a wild-type DNA sequence encoding a native B. clausii protease (Eraser 11).

SEQ ID NO: 7 is the amino acid sequence of the native B. clausii protease (Eraser 11) encoded by SEQ ID NO: 6.

SEQ ID NO: 8 is a DNA sequence comprising a B. amyloliquefaciens BPN′ terminator sequence.

SEQ ID NO: 9 is a DNA sequence comprising a B. subtilis 5′ skfA flanking region (FR) sequence.

SEQ ID NO: 10 is a DNA sequence comprising a B. subtilis 3′ skfA FR sequence.

SEQ ID NO: 11 is a DNA sequence comprising a B. subtilis 5′ aprE FR sequence.

SEQ ID NO: 12 is a DNA sequence comprising a wild-type B. subtilis alrA gene.

SEQ ID NO: 13 is a DNA sequence comprising a B. subtilis 3′ aprE FR sequence.

SEQ ID NO: 14 is a DNA sequence comprising a wild-type B. subtilis IlvE gene coding sequence (CDS).

SEQ ID NO: 15 is the amino acid sequence of the native B. subtilis IlvE protein encoded by SEQ ID NO: 14.

SEQ ID NO: 16 is a DNA sequence comprising a wild-type B. subtilis IlvE promoter.

SEQ ID NO: 17 is a DNA sequence comprising a wild-type B. subtilis IlvE 5′-UTR sequence.

SEQ ID NO: 18 is a DNA sequence comprising a mutant B. subtilis IlvE 5′-UTR sequence.

SEQ ID NO: 19 is a B. subtilis IlvE-forward (FW) primer (DNA) sequence.

SEQ ID NO: 20 is a B. subtilis IlvE-reverse (RV) primer (DNA) sequence.

SEQ ID NO: 21 is a synthetic DNA probed named “IlvE-BBQ”.

SEQ ID NO: 22 is a B. subtilis ftsY-forward (FW) primer (DNA) sequence.

SEQ ID NO: 23 is a B. subtilis ftsY-reverse (RV) primer (DNA) sequence DNA.

SEQ ID NO: 24 is a synthetic DNA probed named “ftsY-BBQ”.

SEQ ID NO: 25 is a DNA sequence comprising the wild-type (WT) B. subtilis IlvE gene, comprising (in the 5′ to 3′ direction) the WT ilvE promoter (SEQ ID NO: 16) operably linked to the WT ilvE 5′-UTR (SEQ ID NO: 17) operably linked to the WT ilvE gene CDS (SEQ ID NO: 14).

SEQ ID NO: 26 is the DNA sequence of a native B. subtilis Hbs promoter region sequence.

SEQ ID NO: 27 is a synthetic DNA construct comprising an upstream (5′) Hbs promoter operably linked to the wild-type IlvE gene.

SEQ ID NO: 28 is a synthetic DNA construct comprising an upstream (5′) Hbs promoter operably linked to the variant IlvE gene.

### DETAILED DESCRIPTION

As described herein, certain embodiments of the disclosure are related to compositions and methods for enhanced protein production in mutant/recombinant Bacillus sp. (host) cells/strains. More particularly, as set forth hereinafter, and further described in the Examples below, recombinant Bacillus cells of the disclosure are particularly useful for the enhanced production of proteins of interest when cultivated under suitable conditions. Thus, certain embodiments of the disclosure provide, inter alia, mutant Bacillus strains comprising variant ilvE gene sequences, recombinant (genetically modified) Bacillus strains comprising variant ilvE gene sequences, mutant and/or recombinant Bacillus strains comprising variant ilvE gene sequences and expressing/producing one or more proteins of interest, methods and compositions for constructing recombinant Bacillus strains comprising variant ilvE gene sequences, expression cassettes encoding proteins of interest, methods and compositions for cultivating recombinant Bacillus strains comprising variant ilvE gene sequences for the enhanced production of proteins of interest and the like.

### I. DEFINITIONS

In view of the recombinant (modified) cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”, “recombining” or generating a “recombined” nucleic acid is generally the assembly of two or more nucleic acid fragments wherein the assembly gives rise to a chimeric gene.

As used herein, the phrases “Gram-positive bacteria”, Gram-positive cells” “Gram-positive bacterial strains”, and/or “Gram positive bacterial cells” have the same meaning as used in the art. For example, Gram-positive bacterial cells include all strains of Actinobacteria and Firmicutes. In certain embodiments, such Gram-positive bacteria are of the classes Bacilli, Clostridia and Mollicutes.

As used herein, “the genus Bacillus” includes all species within the genus “Bacillus”’ as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

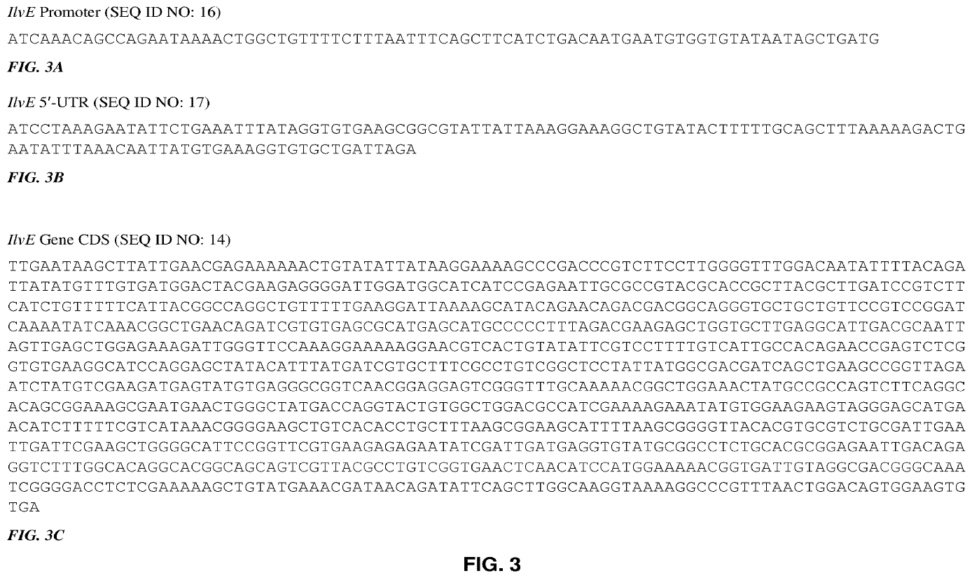

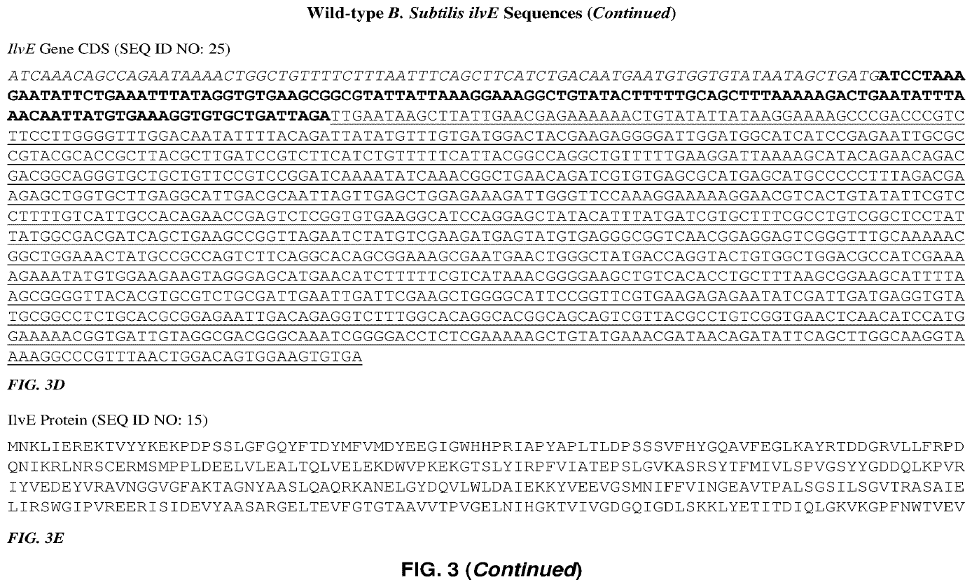

As used herein, a “wild-type B. subtilis ilvE promoter” sequence (abbreviated, “WT ilvE pro”), comprises the nucleotide sequence set forth in SEQ ID NO: 16, as shown in FIG. 3A.

As used herein, a “wild-type B. subtilis ilvE 5′-untranslated region” sequence (abbreviated, “WT ilvE 5′-UTR”), comprises the nucleotide sequence set forth in SEQ ID NO: 17, as shown in FIG. 3B.

As used herein, a “wild-type B. subtilis ilvE gene coding sequence (abbreviated, “gene CDS, CDS or ORF)” comprises the nucleotide set forth in SEQ ID NO: 14, as shown in FIG. 3C.

As used herein, a wild-type ilvE gene comprises the nucleotide set forth in SEQ ID NO: 25, as shown in FIG. 3D.

As used herein, a “native B. subtilis IlvE protein” encoded by a WT B. subtilis gene CDS comprises the amino acid sequence set forth in SEQ ID NO: 15, as shown in FIG. 3E.

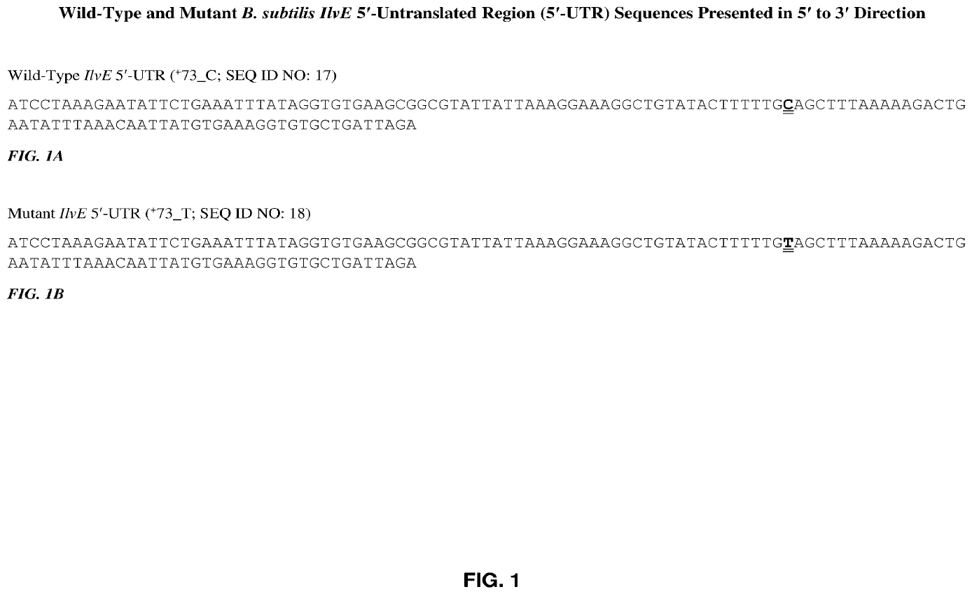

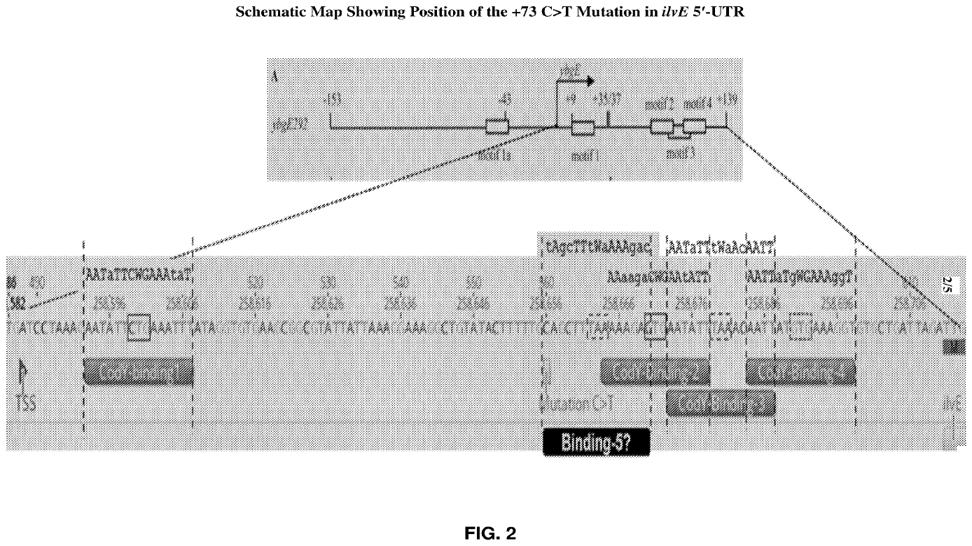

As used herein, a “mutant B. subtilis ilvE 5′-untranslated region” sequence (abbreviated, “mutant ilvE 5′-UTR”), comprises the nucleotide sequence set forth in SEQ ID NO: 18, as shown in FIG. 1. In particular, as shown in FIG. 1, the mutant ilvE 5′-UTR sequence (SEQ ID NO: 18; FIG. 1B) comprises an unexpected single nucleotide polymorphism (SNP) at nucleotide position 73, as compared to the WT B. subtilis ilvE 5′-UTR (SEQ ID NO: 17; FIG. 1A). For example, as presented in FIG. 1, the WT B. subtilis ilvE 5′-UTR sequence comprises a cytosine (C) at nucleotide position 73 (73C; SEQ ID NO: 17), and the mutant ilvE 5′-UTR SNP sequence comprises a thymine (T) at nucleotide position 73 (73T; SEQ ID NO: 18). As presented in FIG. 1, the WT ilvE 5′-UTR sequence (FIG. 1A) is shown with the cytosine (C) at nucleotide position 73 double underlined, and the mutant ilvE 5′-UTR sequence (FIG. 1B) is shown with the thymine (I) at nucleotide position 73 double underlined. As generally shown in the FIG. 2 schematic, nucleotide position 73 of the ilvE 5′-UTR sequence is numbered the from the beginning (+1) of the transcription start site, wherein position 73 may be referred to alternatively as position +73.

As used herein, phrases such as a “B. subtilis P2 promoter” and/or “operably linked to a P2 promoter” particularly refer to the B. subtilis P2 promoter sequence set forth and described in PCT Publication No. WO2020/112609 (incorporated herein by reference in its entirety). More particularly, the B. subtilis P2 promoter is set forth as SEQ ID NO: 40 in PCT Publication No. WO2020/112609.

As used herein, a “host cell” refers to a cell that has the capacity to act as a host or expression vehicle for a newly introduced DNA sequence. Thus, in certain embodiments of the disclosure, the host cells are Gram-positive cells, Bacillus sp. or E. coli cells.

As used herein, the phrases “modified Bacillus cell” and/or “Bacillus daughter cell” refer to a recombinant Bacillus cell that comprises at least one genetic modification which is not present in the parent cell from which the modified cell is derived. In certain embodiments, an “unmodified” Bacillus cell may be referred to as a “control cell”, particularly when being compared with, or relative to, a modified Bacillus cell.

As used herein, when the expression and/or production of a protein of interest (POI) in an “unmodified” (parental or control) cell is being compared to the expression and/or production of the same POI in a “modified” (daughter) cell, it will be understood that the “unmodified” and “modified” cells are grown/cultivated/fermented under the same conditions (e.g., the same conditions such as media, temperature, pH and the like). In certain embodiments, an increased amount of a POI may be an endogenous POI (e.g., native proteases, native amylases, etc.), or a heterologous POI (e.g., recombinant proteases, recombinant amylases, etc.) expressed in a recombinant Bacillus cell of the disclosure.

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, pullulanase activity, cellulase activity, and the like), or total extracellular protein produced as compared to the parental cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence (CDS).

As used herein, the term “coding sequence” (CDS) refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it's capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence (e.g., an ORF) encoding a protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As used herein, the term “introducing”, as used in phrases such as “introducing into a bacterial cell” or “introducing into a Bacillus cell at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the Bacillus sp. chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the Bacillus chromosome a DNA construct is integrated and directs what part of the Bacillus chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, spec^{R}, kan^{R}, ery^{R}, tet^{R}, cmp^{R} and neo^{R}. In some embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids. Other markers useful in accordance with the invention include, but are not limited to auxotrophic markers, such as serine, lysine, tryptophan; and detection markers, such as β-galactosidase.

As defined herein, a host cell “genome”, a bacterial (host) cell “genome”, or a Bacillus sp. (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. In some embodiments, plasmids exist in a parental cell and are lost in the daughter cell.

A used herein, a “transformation cassette” refers to a specific vector comprising a gene (or ORF thereof) and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental B. licheniformis (host) cell is modified (e.g., transformed) by introducing therein one or more “targeting vectors”.

As used herein, the term “protein of interest” or “POI” refers to a polypeptide of interest that is desired to be expressed in a modified B. licheniformis (daughter) host cell, wherein the POI is preferably expressed at increased levels (i.e., relative to the “unmodified” (parental) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest or an endogenous protein of interest relative to the parental cell. In particular embodiments, an increased amount of a protein of interest produced by a modified cell of the disclosure is at least a 0.5% increase, at least a 1.0% increase, at least a 5.0% increase, or a greater than 5.0% increase, relative to the parental cell.

Similarly, as defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexosc oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. The degree of homology between sequences can be determined using any suitable method known in the art (see, e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI); and Devereux et al., 1984). For purposes of the present invention, the degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

### II. MUTANT BACILLUS STRAIN HAVING ENHANCED PROTEIN PRODUCTION AND CARBON YIELD PHENOTYPES

As generally set forth herein and further described below in the Examples, Applicant has identified a mutant B. subtilis strain (named “CZ437”) having an enhanced protein production phenotype. In particular, Applicant performed next-generation sequencing (NGS) on the mutant CZ437 strain to further characterize the enhanced protein productivity phenotype observed, wherein an unexpected single nucleotide polymorphism (SNP) mutation in the wild-type (WT) B. subtilis ilvE 5′-UTR sequence (SEQ ID NO: 17) was identified. For example, the DNA sequences of the WT ilvE 5′-UTR (SEQ ID NO: 17) and mutant ilvE 5′-UTR (SEQ ID NO: 18) are presented in FIG. 1A and FIG. 1B, respectively, wherein the WT ilvE 5′-UTR sequence comprises a cytosine (C) at nucleotide position 73 (SEQ ID NO: 17) and the mutant ilvE 5′-UTR SNP sequence comprises a thymine (T) at nucleotide position 73 (SEQ ID NO: 18).

Without wishing to be bound by any particular theory, mechanism, or mode of operation, Applicant contemplates herein that the unexpected SNP mutation identified in the ilvE 5′-UTR (i.e., SNP C→T) may impact ilvE messenger RNA (mRNA) stability. For example, the ilvE (ybgE) gene is known to encode a branched-chain amino acid aminotransferase that transaminates branched-chain amino acids and ketoglutarate. Berger et al. (2003) demonstrated another function for IlvE in the methionine regeneration pathway, by converting ketomethiobutyrate (KMTB) into methionine. For this reaction, the IvlE amino transferase can use leucine, isoleucine, valine, phenylalanine, and tyrosine as amino donors, while the B. subtilis homologous enzyme YkrV, uses only glutamine as amino donor. As generally described in Mader et al. (2004), the ilvE gene is negatively regulated by the global transcriptional regulator CodY, wherein CodY controls transcription of ilvE by binding to its transcriptional leader (5′-UTR) sequence and serving as a roadblock to the RNA polymerase, resulting in repression of ilvE in presence of casamino acids or free amino acids. As shown/annotated in FIG. 2, the SNP mutation of the ilvE 5′-UTR occurs fifty-five (55) nucleotides (bp) upstream (5′) of the translation start site, which may influence ilvE mRNA stability. In particular, as presented in FIG. 2, the C>T mutation in ilvE 5′-UTR is located near CodY binding motif 2, and at putative weak binding motif 5. As contemplated herein, the weak codY binding motif 5 could potentially mask codY from binding to the motif 2, thereby causing de-repression of ilvE transcription.

Thus, as set forth herein, and further described below in the Examples, Applicant designed, constructed and screened recombinant Bacillus strains expressing a reporter protein (GG36) to further assess the enhanced protein production phenotype identified in the mutant B. subtilis CZ437 strain. More particularly, as set forth in Example 1, two (2) GG36 reporter protein expression cassettes were constructed and introduced into B. subtilis strain A (comprising the mutant ilvE 5′-UTR (SEQ ID NO: 18) and the isogenic B. subtilis strain B (comprising the WT ilvE 5′-UTR (SEQ ID NO:17), wherein strains A and B were fermented under the same conditions using standard fermentation conditions in a large scale (˜14 L) fermentor. As shown in TABLE 1 (Example 1), the relative improvement in carbon yield of the mutant B. subtilis reporter strain A is significantly enhanced as compared to the isogenic reporter strain B.

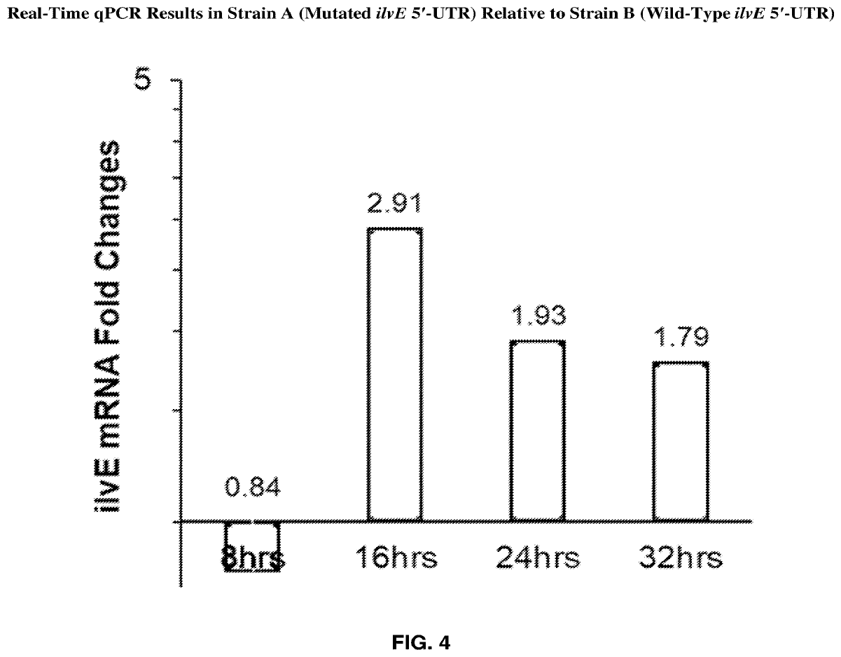

Likewise, as set forth in Example 2, time course samples from B. subtilis strains A and B (expressing GG36 reporter protein) were used for the real time quantitative PCR (RT qPCR) analysis, wherein samples were collected at 8, 16, 24 and 32 hours of fermentation and total RNA was extracted. For example, the fold changes in ilvE mRNA of B. subtilis strain A (mutant ilvE 5′-UTR) versus isogenic B. subtilis strain B (WT ilvE 5′-UTR) at 8, 16, 24 and 32 hours of fermentation are shown in as FIG. 4. More particularly, as presented in FIG. 4, the amount of ilvE mRNA at the 16, 24, and 32 hour fermentation time points is significantly increased 2.91, 1.93 and 1.79 fold, respectively in B. subtilis strain A as compared to the isogenic B. subtilis strain B.

Thus, certain embodiments of the disclosure are related to mutant Bacillus strains comprising variant ilvE gene sequences, recombinant Bacillus strains comprising variant ilvE gene sequences, mutant/recombinant strains comprising variant ilvE gene sequences and expressing/producing one or more proteins of interest, methods and compositions for constructing recombinant Bacillus strains comprising variant ilvE gene sequences, expression cassettes encoding proteins of interest, methods and compositions for cultivating recombinant Bacillus strains comprising variant ilvE gene sequences for the enhanced production of proteins of interest and the like.

### III. RECOMBINANT POLYNUCLEOTIDES AND MOLECULAR BIOLOGY

As generally set forth above, certain embodiments of the disclosure are related to, inter alia, variant ilvE genes, mutant Bacillus strains comprising variant ilvE genes, recombinant (genetically modified) Bacillus strains comprising variant ilvE genes, mutant and/or recombinant Bacillus strains comprising variant ilvE genes and expressing/producing one or more proteins of interest, methods and compositions for constructing recombinant Bacillus strains comprising variant ilvE gene sequences, expression cassettes encoding proteins of interest, methods and compositions for cultivating recombinant Bacillus strains comprising variant ilvE gene sequences for the enhanced production of proteins of interest and the like.

Thus, in one or more embodiments, the disclosure provides recombinant polynucleotides (e.g., vectors, plasmids, expression cassettes, etc.), recombinant (genetically modified) Gram-positive bacterial cells/strains expressing proteins of interest and the like. In certain one or more embodiments, the disclosure provides polynucleotide constructs suitable for introducing into recombinant Gram-positive bacterial cells for the enhanced production of proteins of interest.

In certain embodiments, polynucleotide constructs of the disclosure are referred to as expression cassettes (or expression constructs), wherein the expression cassettes comprise, in the 5′ to 3′ direction and operable combination, at least an upstream (5′) a promoter sequence operably linked to a downstream (3′) gene coding sequence CDS. In certain embodiments, expression cassettes encode one or more proteins of interest (e.g., 5′-[promoter sequence]-[gene coding sequence]-3′; abbreviated, 5′-[pro]-[gene CDS]-3′).

In other embodiments, the disclosure provides ilvE expression cassettes. In certain embodiments, ilvE expression cassettes comprise, in the 5′ to 3′ direction and operable combination, at least an upstream (5′) a promoter sequence operably linked to a variant ilvE 5′-untranslated region (5′-UTR) sequence operably linked to a wild-type ilvE gene CDS (abbreviated, 5′-[pro]-[ilvE* 5′-UTR)]-[WT ilvE CDS]-3′. As abbreviated above, the variant (mutant) ilvE* gene 5′-UTR sequence is presented/shown with an asterisk (*) to distinguish from the wild-type ilvE gene 5′-UTR sequence (i.e., SEQ ID NO: 17).

In certain other embodiments, expression cassettes may comprise one or more DNA sequence elements, including, but not limited to, DNA sequence elements encoding protein/peptide signal (secretion) sequences, DNA sequence elements encoding pro-peptide (pro-region) amino acid residues, DNA sequence elements comprising transcriptional terminator sequences, DNA sequence elements comprising 5′-UTRs, 3′-UTRs, and the like.

Thus, one or more nucleic acid sequences described herein can be generated by using any suitable synthesis, manipulation, and/or isolation techniques, or combinations thereof. For example, one or more polynucleotides described herein may be produced using standard nucleic acid synthesis techniques, such as solid-phase synthesis techniques that are well-known to those skilled in the art. In such techniques, fragments of up to fifty (50) or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method or methods as typically practiced in automated synthetic methods. One or more polynucleotides described herein can also be produced by using an automatic DNA synthesizer. Customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA). Other techniques for synthesizing nucleic acids and related principles are described and known in the art.

Recombinant DNA techniques useful in modification of nucleic acids are well known in the art, such as, for example, restriction endonuclease digestion, ligation, reverse transcription and cDNA production, and polymerase chain reaction (e.g., PCR). One or more polynucleotides described herein may also be obtained by screening cDNA libraries using one or more oligonucleotide probes that can hybridize to or PCR-amplify polynucleotides which encode one or more variants described herein. Procedures for screening and isolating cDNA clones and PCR amplification procedures are well known to those of skill in the art and described in standard references known to those skilled in the art. One or more polynucleotides described herein can be obtained by altering a naturally occurring polynucleotide backbone (e.g., that encodes one or more variant pro-region sequences described herein) by, for example, a known mutagenesis procedure (e.g., site-directed mutagenesis, site saturation mutagenesis, and in vitro recombination). A variety of methods are known in the art that are suitable for generating modified polynucleotides described herein that encode one or more variants described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

As generally set forth above and further described below in the Examples, certain embodiments of the disclosure are related to recombinant (modified) Gram-positive cells capable of producing of heterologous proteins of interest. Certain embodiments are therefore related to methods for constructing such recombinant Gram-positive cells having increased protein production capabilities. In certain embodiments, one or more expression cassettes encoding one or more proteins of intertest are introduced into Gram-positive cells of the disclosure. In exemplary embodiments, the cassettes are integrated into the genome of the cell. Thus, certain embodiments are related to nucleic acid molecules, polynucleotides (e.g., vectors, plasmids, expression cassettes), regulatory elements, and the like, suitable for use in constructing recombinant (modified) Gram-positive host cells.

Accordingly, as presented in the Examples and generally described herein, recombinant cells of the disclosure may be constructed by one of skill using standard and routine recombinant DNA and molecular cloning techniques well known in the art. Methods for genetic modification include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) a gene down-regulation, (f) site specific mutagenesis and/or (g) random mutagenesis.

In certain embodiments, modified cells of the disclosure may be constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments a modified cell is constructed by gene deletion to eliminate or reduce the expression of the gene. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a cell, for example, on a temperature-sensitive plasmid in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art may readily identify nucleotide regions in the gene's coding sequence and/or the gene's non-coding sequence suitable for complete or partial deletion.

In other embodiments, a modified cell is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a Gram-positive cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene encoding a protein of interest can be edited or disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Gram-positive cell and a terminator active in Gram-positive cells, thereby creating a Gram-positive cell Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Gram-positive expression cassette for the gRNA is created by operably linking the DNA encoding the gRNA to a promoter active in Gram-positive cells and a terminator active in Gram-positive cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Gram-positive host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions and selecting for mutant cells exhibiting reduced or no expression of the gene.

PCT Publication No. WO2003/083125 discloses methods for modifying Gram-positive (Bacillus) cells, such as the creation of Bacillus deletion strains and DNA constructs using PCR fusion to bypass E. coli. PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are aware of suitable methods for introducing polynucleotide sequences into bacterial cells (e.g., Gram-negative cells, Gram-positive cells). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art. In some embodiments, resolution of the vector from a host chromosome leaves the flanking regions in the chromosome, while removing the indigenous chromosomal region.

Promoters and promoter sequence regions for use in the expression of genes, coding sequences (CDS), open reading frames (ORFs) and/or variant sequences thereof in Gram-positive cells are generally known on one of skill in the art. Promoter sequences of the disclosure are generally chosen so that they are functional in the Gram-positive cells. For example, promoters useful for driving gene expression in Bacillus cells include, but are not limited to, the B. subtilis alkaline protease (aprE) promoter, the α-amylase promoter (amyE) of B. subtilis, the ax-amylase promoter (amyL) of B. licheniformis, the α-amylase promoter of B. amyloliquefaciens, the neutral protease (nprE) promoter from B. subtilis, a mutant aprE promoter, or any other promoter from B lichemiformis or other related Bacilli. Methods for screening and creating promoter libraries with a range of activities (promoter strength) in Bacillus cells is describe in Publication No. WO2002/14490.

### IV. FERMENTING BACILLUS CELLS FOR THE PRODUCTION OF PROTEINS

As generally described above, certain embodiments are related to compositions and methods for constructing and obtaining Gram-positive cells expressing/producing one or more proteins of interest.

Certain other embodiments of the disclosure are therefore related to methods of producing proteins of interest in Gram-positive cells by fermenting the cells in a suitable medium. Fermentation methods well known in the art can be applied to ferment Gram-positive cells of the disclosure.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch” fermentation system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In certain embodiments, a protein of interest expressed/produced by a Gram-positive cell of the disclosure may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. PROTEINS OF INTEREST

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

For example, in certain embodiments, a mutant or modified (recombinant) Gram-positive cell of the disclosure produces at least about 0.1% more, at least about 0.5% more, at least about 1% more, at least about 5% more, at least about 6% more, at least about 7% more, at least about 8% more, at least about 9% more, or at least about 10% or more of a POI, relative to its unmodified (parental or control) cell.

In certain embodiments, a mutant or modified Gram-positive cell of the disclosure exhibits an increased specific productivity (Qp) of a POI relative the control cell. For example, the detection of specific productivity (Qp) is a suitable method for evaluating protein production. The specific productivity (Qp) can be determined using the following equation:

$$“ Qp = gP / gDCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a mutant or modified Gram-positive cell of the disclosure comprises a specific productivity (Qp) increase of at least about 0.1% more, at least about 0.5% more, at least about 1% more, at least about 5% more, at least about 6% more, at least about 7% more, at least about 8% more, at least about 9% more, or at least about 10% or more, relative to the unmodified (parental/control) cell.

In certain other embodiments, a mutant or modified Gram-positive cell comprises enhanced/increased ilvE messenger RNA (mRNA) levels relative to the ilvE mRNA levels of the control cell. Suitable methods for such mRNA detection and analysis are generally known to one skilled in the art, including, but not limited to real time quantitative PCR (RT qPCR) analysis, RNA sequencing and the like. In certain embodiments, a mutant or modified Gram-positive cell has at least about a 0.1% increase, at least about a 0.5% increase, at least about a 1.0% increase, at least about a 5% increase to about a 10% increase in ilvE mRNA relative to the unmodified (parental/control) cell ilvE mRNA levels.

In certain other embodiments, a mutant or modified Gram-positive cell comprises an enhanced/increased carbon yield phenotype when expressing/producing one or more proteins of interest. In certain embodiments, enhanced/increased carbon yields (i.e., when expressing/producing one or more proteins of interest) may be referred to as enhanced/increased carbon yield efficiencies. For example, product formation by Gram-positive bacterial cells is a biological conversion processes in which the chemical nutrients fed to bacterial cells during fermentation are converted to metabolites.

In certain other embodiments, a variant, modified or mutant Gram-positive cell exhibits an increased total protein yield, wherein total protein yield is defined as the amount of protein of interest produced (g) per total carbohydrate equivalent of batch and carbohydrate fed, relative to the (unmodified/control) parental strain. Thus, as used herein, total protein yield (g/g) may be calculated using the following equation:

$$“ Yf = Tp / Tc ”$$

wherein “Yf” is total protein yield (g/g). “Tp” is the total protein of interest produced during the fermentation (g) and “Tc” is the total carbohydrate equivalent of batch and carbohydrate (g) fed during the fermentation (bioreactor) run. In certain embodiments, the increase in total protein yield of the modified strain (i.e., relative to the control strain) is an increase of at least about 0.1%, at least about 0.5%, at least about 1%, at least about 5%, at least about 6%, at least about 7%, at least about 8%, at least about 9%, or at least about 10% or more as compared to the unmodified (parental) cell.

Total protein carbon yield may also be described as carbon conversion efficiency/carbon yield, for example, as in the percentage (%) of carbon of batch and fed that is incorporated into total protein of interest. Thus, in certain embodiments, a variant Bacillus strain of comprises an increased carbon conversion efficiency (e.g., an increase in the percentage (%) of carbon of batch and fed that is incorporated into total protein), relative to the (control) parental strain. In certain embodiments, the increase in carbon conversion efficiency of the modified strain (i.e., relative to the control strain) is an increase of at least about 0.1%, at least about 0.5%, at least about 1%, at least about 5%, at least about 6%, at least about 7%, at least about 8%, at least about 9%, or at least about 10% or more as compared to the unmodified (parental/control) cell.

Enhanced carbon yields, enhanced carbon yield efficiencies and the like may assessed/determined using routine methods/techniques know to one of skill in the art. In one or more embodiments, a modified cell comprising an enhanced carbon yield is fermented under suitable conditions for the production of proteins of interest, wherein the enhanced carbon yield is the result of nutrients in the fermentation media being more efficiently incorporated into the protein product, demonstrating enhanced protein productivity or yield coefficient (Y).

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

There are various assays known to those of ordinary skill in the art for detecting and measuring activity of intracellularly and extracellularly expressed proteins.

### VI. EXEMPLARY EMBODIMENTS

Non-limiting embodiments of the compositions and methods disclosed herein are as follows:

1. A variant ilvE gene comprising a mutation in the 5′-untranslated region (5′-UTR) of the ilvE gene.

2. The variant ilvE gene of embodiment 1, encoding a functional IlvE protein.

3. The variant ilvE gene of embodiment 1, wherein the mutation is a single nucleotide polymorphism (SNP) mutation in the 5′-UTR of the ilvE gene.

4. The variant ilvE gene of embodiment 4, wherein the mutation in the 5′-UTR is a cytosine (C) to thymine (T) mutation at position 73, wherein the nucleotide positions of the ilvE 5′-UTR are numbered by correspondence with the wild-type (WT) ilvE 5′-UTR sequence of SEQ ID NO: 17.

5. The variant ilvE gene of embodiment 1, comprising at least about 90%, 91%, 92%, 93%, 94%, 95%, 96%, 97%, 98%, 99 or 100% sequence identity to the wild-type B. subtilis ilvE gene of SEQ ID NO: 25.

6. The variant ilvE gene of embodiment 1, wherein the ilvE 5′-UTR comprises at least about 95%, 96%, 97%, 98%, 99% or 100% sequence identity SEQ ID NO: 18 and a thymine (T) at nucleotide position 73.

7. The variant ilvE gene of embodiment 2, wherein the functional IlvE protein comprises at least about 95% sequence identity to the native IlvE protein of SEQ ID NO: 15.

8. The variant ilvE gene of embodiment 1, wherein the WT ilvE gene promoter is replaced with a heterologous promoter.

9. A synthetic ilvE gene construct comprising in the 5′ to 3′ direction, a wild-type ilvE gene promoter or a heterologous promoter sequence operably linked to a mutant ilvE 5′-UTR sequence operably linked to an ilvE gene CDS encoding a functional IlvE protein.

10. The gene construct of embodiment 9, wherein the mutant ilvE 5′-UTR comprises a single nucleotide polymorphism (SNP) mutation in the 5′-UTR of the ilvE gene.

11. The gene construct of embodiment 10, wherein the mutant ilvE 5′-UTR sequence comprises at least 95%, 96%, 97%, 98%, 99% or 100% sequence identity SEQ ID NO: 18 and a thymine (T) at nucleotide position 73.

12. A mutant Bacillus subtilis cell comprising a variant ilvE gene comprising a mutation in the 5′-untranslated region (5′-UTR) of the ilvE gene.

13. The mutant cell of embodiment 12, comprising a single nucleotide polymorphism (SNP) in the 5′-UTR of the ilvE gene, wherein the mutation in the 5′-UTR is a cytosine (C) to thymine (T) mutation at position 73, wherein the nucleotide positions of the ilvE 5′-UTR are numbered by correspondence with the wild-type (WT) ilvE 5′-UTR sequence of SEQ ID NO: 17.

14. The mutant cell of embodiment 12, producing one or more proteins of interest.

15. The mutant cell of embodiment 14, wherein the one or more proteins of interest are selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lectins, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaninases, xylanases, hexose oxidases, and combinations thereof.

16. The mutant cell of embodiment 12, wherein the variant ilvE gene comprises at least about 90%, 91%, 92%, 93%, 94%, 95%, 96%, 97%, 98%, 99 or 100% sequence identity to the wild-type B. subtilis ilvE gene of SEQ ID NO: 25.

17. The mutant cell of embodiment 12, wherein the ilvE 5′-UTR comprises at least about 95%, 96%, 97%, 98%, 99% or 100% sequence identity SEQ ID NO: 18 and a thymine (T) at nucleotide position 73.

18. The mutant cell of embodiment 12, encoding a functional IlvE protein.

19. The mutant cell of embodiment 12, wherein the WT ilvE gene promoter is replaced with a heterologous promoter.

20. The mutant cell of embodiment 19, wherein the heterologous promoter overexpresses the ilvE gene relative to the WT ilvE promoter, when fermented under the same conditions.

21. The mutant cell of embodiment 14, comprising an enhanced carbon yield phenotype as compared to a control cell producing the same one or more proteins of interest and comprising a wild-type ilvE gene, when the mutant and control cells are fermented under the same conditions for the production of the one or more proteins of interest.

22. The mutant cell of embodiment 12, comprising increased ilvE messenger RNA (mRNA) levels as compared to a control cell comprising the WT ilvE gene, when the mutant and control cells are fermented under the same conditions.

23. The mutant cell of embodiment 22, comprising increased ilvE mRNA levels at about sixteen (16) hours of fermentation as compared to a control cell.

24. The mutant cell of embodiment 22, comprising increased ilvE mRNA levels at about twenty-four (24) hours of fermentation as compared to a control cell.

25. The mutant cell of embodiment 22, comprising increased ilvE mRNA levels at about thirty-two (32) hours of fermentation as compared to a control cell.

26. A genetically modified Bacillus subtilis cell derived from a parental cell comprising a wild-type (WT) ilvE gene, wherein the modified cell comprises a variant ilvE gene comprising a mutation in the 5′-UTR sequence of the ilvE gene.

27. The modified cell of embodiment 26, comprising a single nucleotide polymorphism (SNP) mutation in the 5′-UTR sequence of the ilvE gene.

28. The modified cell of embodiment 27, wherein the SNP mutation in the 5′-UTR is a cytosine (C) to thynine (T) mutation at position 73, wherein the nucleotide positions of the 5′-UTR are numbered by correspondence with the WT ilvE 5′-UTR sequence of SEQ ID NO: 17.

29. The modified cell of embodiment 26 producing one or more proteins of interest.

30. The modified cell of embodiment 28, wherein the one or more proteins of interest are selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lectins, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaninases, xylanases, hexose oxidases, and combinations thereof.

31. The modified cell of embodiment 26, wherein the variant ilvE gene comprises at least about 90%, 91%, 92%, 93%, 94%, 95%, 96%, 97%, 98%, 99 or 100% sequence identity to the wild-type B. subtilis ilvE gene of SEQ ID NO: 25.

32. The modified cell of embodiment 26, wherein the ilvE 5′-UTR comprises at least about 95%, 96%, 97%, 98%, 99% or 100% sequence identity SEQ ID NO: 18 and a thynine (T) at nucleotide position 73.

33. The modified cell of embodiment 26, encoding a functional IlvE protein.

34. The modified cell of embodiment 26, wherein the WT ilvE gene promoter is replaced with a heterologous promoter.

35. The modified cell of embodiment 34, wherein the heterologous promoter overexpresses the ilvE gene relative to the WT ilvE promoter when fermented under the same conditions.

36. The modified cell of embodiment 29, comprising an enhanced carbon yield phenotype as compared to a control cell producing the same one or more proteins of interest and comprising a wild-type ilvE gene, when the modified and control cells are fermented under the same conditions for the production of the one or more proteins of interest.

37. The modified cell of embodiment 26, comprising increased ilvE messenger RNA (mRNA) levels as compared to a control cell comprising the WT ilvE gene, when the modified and control cells are fermented under suitable conditions.

38. The modified cell of embodiment 37, comprising increased ilvE mRNA levels at about sixteen (16) hours of fermentation as compared to a control cell.

39. The modified cell of embodiment 37, comprising increased ilvE mRNA levels at about twenty-four (24) hours of fermentation as compared to a control cell.

40. The modified cell of embodiment 37, comprising increased ilvE mRNA levels at about thirty-two (32) hours of fermentation as compared to a control cell.

41. A method for increasing ilvE messenger RNA (mRNA) levels in a recombinant Bacillus subtilis cell comprising (a) obtaining a parental B. subtilis cell comprising a wild-type (WT) ilvE gene and replacing the WT ilvE gene with a variant ilvE gene comprising a mutation in 5′-untranslated region (5′-UTR) of the ilvE gene, and (b) fermenting the parental and modified cells for at least about sixteen (16) hours under the same conditions, wherein the modified cell comprises increased levels of ilvE mRNA as compared to the parental cell.

42. The method of embodiment 41, wherein the variant ilvE gene comprises a single nucleotide polymorphism (SNP) mutation in the ilvE 5′-UTR sequence.

43. The method of embodiment 42, wherein the SNP mutation in the 5′-UTR is a cytosine (C) to thymine (T) mutation at position 73, wherein the nucleotide positions of the ilvE 5′-UTR are numbered by correspondence with the wild-type (WT) ilvE 5′-UTR sequence of SEQ ID NO: 17.

44. A method for increasing ilvE messenger RNA (mRNA) levels in a modified Bacillus subtilis cell comprising: (a) obtaining a parental B. subtilis comprising a wild-type (WT) ilvE gene and mutating the 5′-untranslated region (5′-UTR) of the WT ilvE gene to obtain a modified B. subtilis cell comprising a mutation in 5′-untranslated region (5′-UTR) of the ilvE gene, and (b) fermenting the parental and modified cells for at least about sixteen (16) hours under the same conditions, wherein the modified cell comprises increased levels of ilvE mRNA as compared to the parental cell.

45. The method of embodiment 44, wherein the variant ilvE gene comprises a single nucleotide polymorphism (SNP) mutation in the ilvE 5′-UTR sequence.

46. The method of embodiment 45, wherein the SNP mutation in the 5′-UTR is a cytosine (C) to thymine (T) mutation at position 73, wherein the nucleotide positions of the ilvE 5′-UTR are numbered by correspondence with the WT ilvE 5′-UTR sequence of SEQ ID NO: 17.

47. A method for increasing ilvE messenger RNA (mRNA) levels in a modified Bacillus subtilis cell comprising: (a) obtaining a parental B. subtilis comprising a wild-type (WT) ilvE gene and mutating the 5′-untranslated region (5′-UTR) of the WT ilvE gene to obtain a modified B. subtilis cell comprising a variant ilvE gene, and (b) fermenting the parental and modified cells for at least about sixteen (16) hours under the same conditions, wherein the modified cell comprises increased levels of ilvE mRNA as compared to the parental cell.

48. The method of embodiment 47, wherein the variant ilvE gene comprises a single nucleotide polymorphism (SNP) mutation in the ilvE 5′-UTR sequence.

49. The method of embodiment 48, wherein the SNP mutation in the 5′-UTR is a cytosine (C) to thymine (T) mutation at position 73, wherein the nucleotide positions of the ilvE 5′-UTR are numbered by correspondence with the WT ilvE 5′-UTR sequence of SEQ ID NO: 17.

50. A method increasing carbon yield of heterologous proteins produced in a modified Bacillus subtilis cell comprising (a) obtaining or constructing a parental B. subtilis cell producing a heterologous protein of interest (POI), and replacing the wild-type (WT) ilvE gene with a variant ilvE gene and (b) fermenting the parental and modified cells for at least about sixteen (16) hours under suitable the same conditions for the production of the POI, wherein the modified cell comprises an increased carbon yield efficiency of the POI produced as compared to the parental cell.

51. The method of embodiment 50, wherein the variant ilvE gene comprises a single nucleotide polymorphism (SNP) mutation in the ilvE 5′-UTR sequence.

52. The method of embodiment 51, wherein the SNP mutation in the 5′-UTR is a cytosine (C) to thymine (T) mutation at position 73, wherein the nucleotide positions of the ilvE 5′-UTR are numbered by correspondence with the WT ilvE 5′-UTR sequence of SEQ ID NO: 17.

53. A method increasing carbon yield of heterologous proteins expressed/produced in a modified Bacillus subtilis cell comprising: (a) obtaining a parental B. subtilis comprising a wild-type (WT) ilvE gene and mutating the 5′-untranslated region (5′-UTR) of the WT ilvE gene to obtain a modified B. subtilis cell comprising a variant ilvE gene and (b) fermenting the parental and modified cells for at least about sixteen (16) hours under the same conditions, wherein the modified cell comprises an increased carbon yield efficiency of the POI produced as compared to the parental cell.

54. The method of embodiment 53, wherein the variant ilvE gene comprises a single nucleotide polymorphism (SNP) mutation in the ilvE 5′-UTR sequence.

55. The method of embodiment 54, wherein the SNP mutation in the 5′-UTR is a cytosine (C) to thymine (T) mutation at position 73, wherein the nucleotide positions of the ilvE 5′-UTR are numbered by correspondence with the WT ilvE 5′-UTR sequence of SEQ ID NO: 17.

56. The method of any one of embodiments 41-55, wherein the variant ilvE gene comprises at least about 90%, 91%, 92%, 93%, 94%, 95%, 96%, 97%, 98%, 99 or 100% sequence identity to the wild-type B. subtilis ilvE gene of SEQ ID NO: 25.

57. The method of any one of embodiments 41-55, wherein the mutated ilvE 5′-UTR comprises at least about 95%, 96%, 97%, 98%, 99% or 100% sequence identity SEQ ID NO: 18 and a thymine (T) at nucleotide position 73.

58. The method of any one of embodiments 41-55, wherein the WT ilvE gene promoter is replaced with a heterologous promoter.

59. The method of embodiment 58, wherein the heterologous promoter overexpresses the ilvE gene relative to the WT ilvE promoter when fermented under the same conditions.

60. The method of any one of embodiments 41, 44, 47, 50 or 55, wherein the heterologous POI is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lectins, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

61. The method of any one of embodiments 41-55, wherein the variant ilvE gene encodes a functional IlvE protein.

62. The method of embodiment 41 or embodiment 44, wherein the modified cell comprises increased ilvE messenger RNA (mRNA) levels relative to the control cell when fermented under the same conditions for at least about sixteen (16) hours.

63. The method of embodiment 41 or embodiment 44, wherein the modified cell comprises increased ilvE mRNA levels relative to the control cell when fermented under the same conditions for at least about twenty-four (24) hours.

64. The method of embodiment 41 or embodiment 44, wherein the modified cell comprises increased ilvE mRNA levels relative to the control cell when fermented under the same conditions for at least about thirty-two (32) hours.

65. The method of embodiment 50 or embodiment 53, wherein the modified cell comprises an increased carbon yield efficiency relative to the parental cell when fermented under the same conditions for at least about sixteen (16) hours.

66. The method of embodiment 50 or embodiment 53, wherein the modified cell comprises an increased carbon yield efficiency relative to the parental cell when fermented under the same conditions for at least about twenty-four (24) hours.

67. The method of embodiment 50 or embodiment 53, wherein the modified cell comprises an increased carbon yield efficiency relative to the parental cell when fermented under the same conditions for at least about thirty-two (32) hours.

### EXAMPLES

Certain embodiments of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Construction of a Bacillus Protease Reporter Strain

As generally described above, Applicant has identified a mutant B. subtilis cell (strain CZ437) having an enhanced protein production phenotype. In particular, Applicant performed next-generation sequencing (NGS) on the mutant CZ437 strain to further characterize the enhanced protein productivity phenotype observed, wherein an unexpected SNP in the B. subtilis ilvE 5′-UTR sequence was identified. For example, the DNA sequences of the WT ilvE 5′-UTR (SEQ ID NO: 17) and mutant ilvE 5′-UTR (SEQ ID NO: 18) are presented in FIG. 1A and FIG. 1B, respectively. As shown in FIG. 1, the WT ilvE 5′-UTR sequence (SEQ ID NO: 17; FIG. 1A) comprises a cytosine (C) at nucleotide position 73 (⁺73C), and the mutant ilvE 5′-UTR sequence (SEQ ID NO: 18; FIG. 1B) comprises a thymine (T) at nucleotide position 73 (+73T). In the instant example, Applicant constructed a recombinant B. subtilis strain expressing a heterologous reporter (GG36) protein to evaluate the mutant ilvE 5′-UTR sequence (SEQ ID NO: 18) as related to the enhanced protein production phenotype identified in the mutant B. subtilis CZ437 strain. In particular, the DNA fragments described herein were assembled using standard molecular biology techniques and were used as a template to develop linear DNA expression cassettes for integration into B. subtilis strains described herein.

### A. Construction of Reporter Protein Expression Cassettes.

The construction of the reporter protein cassette was performed as follows: a first (1V) DNA fragment containing the 5′ skfA flanking region (FR) sequence (5′ skfA FR; SEQ ID NO: 9) of B. subtilis was operably linked to an expression cassette comprising an upstream (5′) B. subtilis P2 promoter operably linked to a DNA sequence comprising a wild-type B. subtilis aprE 5′-untranslated region (5′-UTR; SEQ ID NO: 1) operably linked to a DNA sequence encoding a wild-type B. subtilis aprE signal sequence (SEQ ID NO: 2) operably linked to a DNA sequence encoding a variant B. lentus pro-peptide sequence (SEQ ID NO: 4) operably linked to a DNA sequence encoding a mature (GG36) subtilisin reporter (SEQ ID NO: 6) operably linked to a BPN′ terminator sequence (SEQ ID NO:8) which was operably linked to a 3′ skfH FR sequence (3′ skfH FR; SEQ ID NO:10).

A second (2^{nd}) DNA fragment comprising a 5′ yhfN flanking region (FR) sequence located in the chromosomal region of the B. subtilis 5′ aprE flanking region (FR) sequence (5′ aprE FR; SEQ ID NO: 11) was operably linked to an expression cassette comprising an upstream (5′) B. subtilis P2 promoter operably linked to a DNA sequence comprising a wild-type B. subtilis aprE 5′-UTR (SEQ ID NO: 1) operably linked to a DNA encoding a wild-type B. subtilis aprE signal sequence (SEQ ID NO: 2) operably linked to a DNA sequence encoding a variant B. lentus pro-peptide sequence (SEQ ID NO: 4) operably linked to a DNA sequence encoding a mature (GG36) subtilisin reporter (SEQ ID NO: 6) operably linked to a BPN′ terminator (SEQ ID NO. 8). The GG36 subtilisin reporter expression cassette was further ligated to the B. subtilis alanine racemase (alrA) gene (SEQ ID NO: 12) and to a 3′ aprE FR sequence (3′ aprE FR; SEQ ID NO: 13).

B. Mutagenesis of ilvE Transcriptional Leader Sequence

As briefly set forth above, a cytosine (C) to thymine (T) mutation at position 73 of ilvE 5′-UTR was introduced in the genome of B. subtilis using random strain mutagenesis. In particular, the 1^{st} and 2^{nd} cassettes described above were integrated into the B. subtilis strain comprising the position 73 C to T (SNP) mutation in the ilvE 5′-UTR (reporter Strain A; 73T) and the isogenic B. subtilis strain comprising wild-type ilvE 5′-UTR (reporter Strain B; 73C).

More particularly, B. subtilis strain A (mutant ilvE 5′-UTR; SEQ ID NO: 18) and isogenic B. subtilis strain B (WT ilvE 5′-UTR; SEQ ID NO: 17) were fermented using standard fermentation conditions in a large scale (˜14 L) fermentor. As presented below in TABLE 1. the relative improvement in carbon yield of the mutant B. subtilis reporter strain A is significantly enhanced as compared to the isogenic B. subtilis reporter strain B.

TABLE 1
Relative Improvement in Carbon Yield of B. subtilis
Reporter Strain A Comprising the Mutated ilvE 5′-UTR

| | % of Carbon Yield Improvement |
|---|---|
| GG36 Protease | 0.8 +/− 0.2 |

C. Replacement of Wild-Type ilvE Promoter with Heterologous Hbs Promoter

Two (2) cassettes for expression of the ilvE amino acid aminotransferase were constructed using conventional molecular biology techniques. More specifically, the ilvE expression cassettes comprise an upstream (5′) wild-type (WT) B. subtilis hbs promoter (Phbs) region sequence (SEQ ID NO: 26) operably linked to a downstream (3′) DNA sequence comprising either the WT ilvE transcriptional leader (WT 5′-UTR; SEQ ID NO: 17) or a mutant ilvE transcriptional leader (mutant 5′-UTR; SEQ ID NO: 18) operably linked to a downstream (3′) WT ilvE gene CDS (SEQ ID NO: 14). Thus, the hbs promoter region (SEQ ID NO: 26) drives expression of both cassettes, wherein the DNA sequence of the WT ilvE gene cassette is set forth in SEQ ID NO: 27, and the sequence of the mutant ilvE gene cassette is set forth on SEQ ID NO:28. More particularly, the cassettes (SEQ ID NO: 27 or SEQ ID NO: 28) were integrated into the spoIIIAA genomic locus of a parental B. subtilis strain comprising two GG36 reporter protein cassettes.

As generally shown below in TABLE 2. B. subtilis strains overexpressing the ilvE gene under control of the hbs promoter and comprising the WT ilvE 5′-UTR (strain CZ477) or the mutated ilvE 5′-UTR (strain CZ488) with were fermented in a large scale (˜14 L) bioreactor under standard fermentation conditions and compared to strain CZ450 comprising the WT ilvE promoter and the WT ilvE 5′-UTR. As presented in TABLE 2, both strains with the overexpression of the ilvE gene showed an increase in carbon efficiency compared to the strain with the WT ilvE promoter.

TABLE 2
Relative Improvement in Carbon Yield of B. subtilis Reporter
Strains Overexpressing the ilvE Gene Using hbs Promoter

| | % of Carbon Yield Improvement | % of Carbon Yield Improvement |
|---|---|---|
| | CZ477 | CZ488 |
| GG36 Protease | 1.15 +/− 0.2 | 1.16 +/− 0.2 |

Thus, in one or more embodiments of the disclosure, as demonstrated in the instant example, a highly expressed heterologous promoter region sequence (e.g., hbs promoter, etc.) may be used to overexpress a variant ilvE gene (or an ilvE gene expression construct thereof) comprising a WT ilvE 5′-UTR sequence operably linked to downstream WT ilvE gene CDS (or a variant ilvE gene CDS thereof encoding a functional ilvE protein) and/or to overexpress a variant ilvE gene (or an ilvE gene expression construct thereof) comprising a mutated ilvE 5′-UTR sequence operably linked to downstream WT ilvE gene CDS (or a variant ilvE gene CDS thereof encoding as functional ilvE protein). Accordingly, such methods, genetic elements, expressions constructs, modified cells comprising enhanced protein production phenotypes, modified cells comprising enhanced carbon yield phenotypes and the like are particularly beneficial in the expression/production of proteins of interests when cultivated under suitable conditions.

### Example 2

### Real Time Quantitative PCR RNA Analysis

In the instant example, time course samples from B. subtilis reporter strains A (mutant ilvE 5′-UTR) and B (WT ilvE 5′-UTR) were used for the real time quantitative PCR (RT qPCR) analysis. For example, samples were collected at 8, 16, 24 and 32 hours of fermentation and total RNA extraction was carried out. In particular, the extracted RNA samples were treated with DNase-I to remove genomic DNA from the samples, and then cDNA was synthesized with Transcriptor First Strand cDNA Synthesis Kit (Roche). Subsequently, 1000-fold diluted cDNA from each sample was used as template for qPCR, and the ftsY gene was used as housekeeping gene for data normalization. For example, sequence specific ilvE forward (SEQ ID NO: 19) and reverse (SEQ ID NO: 20) primers, and ilvE probe (SEQ ID NO: 21) were used for the amplification of a sequence within the ilvE gene. Likewise, sequence specific ftsY forward (SEQ ID NO: 22) and reverse (SEQ ID NO: 23) primers, and ftsY probe (SEQ ID NO: 24) were used for the amplification of a sequence within the ftsY gene.

In particular, the RT qPCR time course experiment data are presented in FIG. 4, wherein the 2 ΔΔC_{T} method (Livak and Schmittgen, 2001) was used to calculate the log-fold changes between the housekeeping ftsY gene and the ilvE gene. As shown in FIG. 4, the bars and values represent the fold changes in ilvE mRNA of B. subtilis strain A (mutant ilvE 5′-UTR) versus the isogenic B. subtilis strain B (WT ilvE 5′-UTR). In particular, with the exception of the first time point at 8 hours of fermentation (i.e., where the amount of ilvE mRNA only differs 0.84 fold), the amount of ilvE mRNA at the 16, 24 and 32 hour fermentation time points is significantly increased 2.91, 1.93 and 1.79 fold, respectively (FIG. 4) in B. subtilis strain A (mutant ilvE 5′-UTR) compared to the isogenic B. subtilis strain B (WT ilvE 5′-UTR). Based on the foregoing, the results presented and described herein demonstrate that the C to T SNP (SEQ ID NO: 18) identified herein significantly increases the level of ilvE mRNA.

### REFERENCES

PCT Publication No. WO2002/14490
PCT Publication No. WO2003/083125
PCT Publication No. WO2020/112609
Ausubel et al., “Current Protocols in Molecular Biology”, published by Greene Publishing Assoc. and Wiley-Interscience (1987).
Belitsky and Sonenshein, “Roadblock repression of transcription by Bacillus subtilis CodY”, J. Mol. Biol. 26; 411(4):729-743, 2011.
Berger et al., “Methionine Regeneration and Aminotransferases in Bacillus subtilis, Bacillus cereus, and Bacillus anthracis”, J. Bacteriol. 185(8):2418-2431, 2003.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Earl et al., “Ecology and genomics of Bacillus subtilis”, Trends in Microbiology., 16(6):269-275, 2008.
Livak and Schmittgen, “Analysis of Relative Gene Expression Data Using Real-Time Quantitative PCR and the 2⁻ΔΔC_{T} Method”, Methods. Vol. 25, pp. 402-408, 2001.
Mäder et al., “Transcriptional organization and posttranscriptional regulation of the Bacillus subtilis branched-chain amino acid biosynthesis genes”, J Bacteriol. 186(8):2240-2252, 2004.
Olempska-Beer et al., “Food-processing enzymes from recombinant microorganisms—a review”’ Regul. Toxicol. Pharnacol., 45(2):144-158, 2006.
Sambrook et al., “Molecular Cloning: A Laboratory Manual” Cold Spring Harbor Laboratory: Cold Spring Harbor, N. Y. (1989), (2001) and (2012).
Van Dijl and Hecker, “Bacillus subtilis: from soil bacterium to super-secreting cell factory”, Microbial Cell Factories, 12(3), 2013.

1. (canceled)

2. (canceled)

3. (canceled)

4. (canceled)

5. A genetically modified Bacillus subtilis cell derived from a parental cell comprising a wild-type (WT) ilvE gene, wherein the modified cell comprises an introduced synthetic ilvE gene comprising a heterologous gene promoter operably linked to a mutant ilvE 5′-UTR sequence comprising SEQ ID NO: 18 operably linked to a WT ilvE gene CDS.

6. (canceled)

7. The modified cell of claim 5, producing one or more proteins of interest.

8. The modified cell of claim 5, wherein the heterologous gene promoter overexpresses the ilvE gene.

9. A method for increasing ilvE messenger RNA (mRNA) levels in a modified Bacillus subtilis cell comprising:
(a) obtaining a parental B. subtilis cell comprising a wild-type (WT) ilvE gene and replacing the WT ilvE gene with a synthetic ilvE gene comprising a heterologous gene promoter operably linked to a mutant ilvE 5′-UTR sequence comprising SEQ ID NO: 18 operably linked to a WT ilvE gene CDS, and
(b) fermenting the parental and modified cells for at least about sixteen (16) hours under the same conditions,
wherein the modified cell comprises increased levels of ilvE mRNA as compared to the parental cell.

10. The method of claim 9, wherein the synthetic ilvE gene comprises at least 90% identity to the ilvE gene of SEQ ID NO: 25 and comprises a thymine (T) at nucleotide position 73 of the ilvE 5′-UTR.

11. A method increasing carbon yield of heterologous proteins produced in a modified Bacillus subtilis cell comprising:
(a) obtaining or constructing a parental B. subtilis cell producing a heterologous protein of interest (POI) and introducing into the cell a synthetic ilvE gene comprising a heterologous gene promoter operably linked to a mutant ilvE 5′-UTR sequence comprising SEQ ID NO: 18 operably linked to a WT ilvE gene CDS, and
(b) fermenting the parental and modified cells for at least about sixteen (16) hours under suitable the same conditions for the production of the POI,
wherein the modified cell comprises an increased carbon yield efficiency of the POI produced as compared to the parental cell.

12. The method of claim 11, wherein the synthetic ilvE gene comprises at least 90% identity to the ilvE gene of SEQ ID NO: 25 and comprises a thymine (T) at nucleotide position 73 of the ilvE 5′-UTR.
